(19) United States
(12) Patent Application Publication
(10) Pub. No.: US 2014/0080887 A1
(43) Pub. Date: Mar. 20, 2014

(54) METHODS FOR IMPROVING LINEAR GROWTH RESPONSE IN CHILDREN

(71) Applicants: Amy L. Heaton, Salt Lake City, UT (US); Mitchell K. Friedlander, Salt Lake City, UT (US); Dennis Gay, Salt Lake City, UT (US)

(72) Inventors: Amy L. Heaton, Salt Lake City, UT (US); Mitchell K. Friedlander, Salt Lake City, UT (US); Dennis Gay, Salt Lake City, UT (US)

(73) Assignee: Basic Research L.L.C., Salt Lake City, UT (US)

(21) Appl. No.: 13/623,096

(22) Filed: Sep. 19, 2012

(51) Int. Cl. A61K 31/401; A61P 3/02

(52) U.S. Cl. 514/423

(57) ABSTRACT

Embodiments of the invention generally relate to methods and supplements for increasing linear growth in borderline GH deficient and non-GH deficient children.

### TECHNICAL FIELD

Embodiments of the invention generally relate to methods and supplements for improving linear growth response in borderline growth hormone deficient and nongrowth hormone deficient children.

### BACKGROUND

Children with growth failure, normal growth hormone responses to stimulation tests, and low somatomedin levels are being recognized with increasing frequency. However, the use of biosynthetic growth hormone (GH) to treat children with idiopathic, familial, or constitutional short stature (hereafter referred to as idiopathic short stature) is controversial. There is ongoing debate among the medical community, third-party payers, and families of affected children about the appropriateness and effectiveness of treatment. More than 1 million children in the United States are potential candidates for GH treatment and are thus affected by decisions about GH use. Corresponding annual expenditures for GH potentially range from $196 million to $18 billion, depending on the criteria for treatment. Although historically reserved and approved by the Food and Drug Administration for treatment of short stature in children with classic GH deficiency, Turner syndrome, renal failure, or Prader-Willi syndrome, GH therapy has been suggested for many other conditions (including idiopathic short stature), and the literature suggests that its use in such children is expanding. The lack of clear data on effectiveness of GH therapy in idiopathic short stature is particularly important. Differing perceptions of GH effectiveness result in marked variation among physicians about recommending GH therapy, and there are striking inconsistencies among third-party payer policies for coverage of GH.

Children with idiopathic short stature constitute the largest population of potential pediatric candidates for GH. For this reason, together with controversy about the distinction between disorder and the bounds of natural variation, idiopathic short stature represents a major threshold in the expansion of nontraditional use of GH. Despite several studies, the effectiveness of GH in increasing growth for children with idiopathic short stature is not clear. Interpretation of the literature has been hampered by studies involving small numbers of participants, variation in outcome measures (eg, short term vs long term, and height vs growth velocity), differing treatment effects reported, and absence of structured synthesis of data. In addition, ethical and practical issues, such as long-term daily injections of placebo to children, have made randomized controlled trials of GH challenging.

The controversies surrounding GH use, the vast number of children affected by decisions about GH, and the high cost of treatment, underscores the importance of providing alternative methods of providing non-GH therapies to increase linear growth in borderline GH deficient and non-GH deficient children. It would be desirable to provide a nutritional supplement to provide such increase in linear growth in children.

### BRIEF SUMMARY OF THE INVENTION

The present invention is generally drawn to a nutritional supplement and method of using the same. The supplement includes an amino acid secretagogue composition, which, taken orally, stimulates the pituitary gland to release hGH.

A particular embodiment of the present disclosure relates to administration of an oral nutritional supplement that includes L-arginine, Oxo-proline, and L-lysine to increase linear growth in borderline GH deficient and non-GH deficient children.

Another particular embodiment relates to administration of an oral nutritional supplement to borderline GH deficient and non-GH deficient children that includes L-arginine hydrochloride, Oxo-proline, L-lysine hydrochloride, N-acetyl-L-cysteine, L-glutamine; and schizonepeta powder to increase linear growth.

Other embodiments are drawn to methods of increasing linear growth response in children that include orally administering the disclosed nutritional supplement to borderline GH deficient and non-GH deficient children.

### DETAILED DESCRIPTION OF THE INVENTION

The present invention relates to a nutritional supplement for use by a human being. The present invention is drawn to a nutritional supplement and method of using the same. The nutritional supplement is an amino acid secretagogue composition, which, taken orally, increases linear growth response in borderline GH deficient and non-GH deficient children. The supplement of the present invention works as a dietary supplement by assisting the body's own ability to increase linear growth naturally in a manner which is safe and effective, as well as being affordable.

A particular embodiment of the present disclosure relates to an oral nutritional supplement that includes L-arginine, Oxo-proline, L-lysine, and, optionally, cysteine. The supplement may additionally include glutamine and/or schizonepeta powder. In particular embodiments, the L-arginine hydrochloride, Oxo-proline, and the L-lysine hydrochloride may be present in an amount of 2.9 grams. The L-arginine hydrochloride and the Oxo-proline may be present in a 1:1 weight ratio. The L-arginine hydrochloride and the Oxo-proline may also be present in a 1:1 mole ratio. The cysteine can be n-acetyl L-cysteine and the glutamine may be L-glutamine. The nutritional supplement may be in any acceptable and known oral formulation, such as powder, tablet, capsule, liquid, or wafer form.

Another particular embodiment relates to an oral nutritional supplement that consists essentially of L-arginine hydrochloride, Oxo-proline, L-lysine hydrochloride, N-acetyl-L-cysteine, L-glutamine; and schizonepeta powder. In particular embodiments, the L-arginine hydrochloride, Oxo-proline, and L-lysine hydrochloride may be present in an amount of 2.9 grams. The L-arginine hydrochloride and the Oxo-proline may be present in a 1:1 weight ratio. The L-arginine hydrochloride and the Oxo-proline may also be present in a 1:1 mole ratio. The cysteine can be n-acetyl L-cysteine and the glutamine may be L-glutamine. The nutritional supplement may be in any acceptable and known oral formulation, such as powder, tablet, capsule, liquid, or wafer form.

Other embodiments are drawn to methods of increasing linear growth response in borderline GH deficient and non-GH deficient children that include orally administering the disclosed nutritional supplement to a human being. Particular embodiments of the invention relate to oral administration of the disclosed nutritional supplement to a child that is borderline GH deficient or non-GH deficient. The nutritional supplement may be administered from one to three times daily or, alternatively, may be administered every other day, or may be administered once a week. In particular embodiments, the nutritional supplement may be administered on an empty stomach.

In accordance with the “consist essentially of” and “consisting essentially of” language, the nutritional supplement of the third embodiments is essentially limited to the aforementioned ingredients and does not include any additional active ingredients intended to add nutritional content (e.g., vitamins, minerals, etc.), but may include additional ingredients not intended to add nutritional content such as ingredients intended to fulfill a non-nutritional purpose (e.g., coloring, fillers, flavoring, an ingredient for maintaining the structural form, etc.).

Each ingredient of the nutritional supplement of the present invention may be prepared in accordance with any method known to one of ordinary skill in the art. Alternatively, each ingredient may be obtained in a fully prepared from a commercially available source.

The nutritional supplement of the present invention may be in any suitable oral administration form, including but not limited to: a chewable form, a liquid form, a spray form, a capsule form, a suppository form, dissolvable wafer, and a powder form.

Irrespective of the structural form of the nutritional supplement, the ingredients of the nutritional supplement may be distributed homogeneously or nonhomogeneously within the nutritional supplement.

The nutritional supplement of the present invention may be ingested on a regular basis, such as a daily or weekly intake at a dosage tailored to an individual's needs; i.e., the nutritional supplement is to be taken regularly as multiples (1×, 2×, etc.) of the structural units (pills, tablets, capsules, etc.) in accordance with the needs of the individual. Alternatively, the nutritional supplement of the present invention may be ingested on an as-needed basis at a dosage tailored to the individual's needs. Medical or nutritional counseling may be beneficial for arriving at a desirable or optimal dosage tailored to the individual's needs.

The combination of types of amino acids, mass ranges, and specific formulations have been selected to be synergistically balanced and of adequate quantity to achieve the desired linear growth effect. Improper combinations of the amino acids may be ineffective. The component amino acids are synergistic in the sense that several of them when combined together, synergistically increase the linear growth in children that are borderline GH deficient and non-GH deficient. The combination was also chosen to reduce or inhibit chemical combination or reaction between the amino acids.

### EXAMPLES

Linear growth responses to treatment with the oral nutritional supplement are compared between GHD short children and non-GHD short children. Children are selected and classified as GHD and non-GHD. Both groups of children are then given the oral supplement for 6 months at standard doses to determine the linear growth responses to the supplement. The subject children meet the following criteria: (a) height between the 5^{th} and the 95^{th} percentile; (b) body weight between 95 and 115% of ideal; (c) no known chronic medical condition; (d) no drug treatment during the period of the study; (e) normal history and physical examination within 6 months before the beginning of the study; and (f) normal hemogram and urinalysis with in 6 months before the beginning of the study.

The children are measured every two weeks on wall-mounted stadiometers. The measurements are continued as long as the children remain in the study. The group of non-GHD children have average responses that are 50-100% as great as in the GHD group of children.

Satisfactory responses are determined as a 5 cm/yr or greater increment in growth velocity to oral nutritional supplement treatment. Linear growth responses by the non-GHD short children resemble that of the GHD patients. The data will support the conclusion that linear growth response to the oral nutritional supplement is not a unique feature of GHD children but can be elicited in many non-GHD children.

While embodiments of the present invention have been described herein for purposes of illustration, many modifications and changes will become apparent to those skilled in the art. Accordingly, the appended claims are intended to encompass all such modifications and changes as fall within the true spirit and scope of this invention.

1. A method of increase linear growth in borderline GH deficient and non-GH deficient children, comprising:
providing an oral nutritional supplement, comprising:
L-arginine;
Oxo-proline; and
L-lysine; and
orally administering the nutritional supplement to a borderline GH deficient or non-GH deficient child.

2. The method of claim 1, wherein the L-arginine hydrochloride is present in an amount from 0.1 to 6 moles, and the oxo-proline is present in an amount from 0.1 to 8 moles.

3. The method of claim 1, wherein the L-lysine is present in an amount from 0.1 to 12 moles.

4. The method of claim 1, wherein the L-arginine is present in an amount from 2.5 to 4.5 moles, and the oxo-proline is present in an amount from 4 to 6 moles.

5. (canceled)

6. The method of claim 1, wherein the nutritional supplement is present in an amount of 2.9 grams.

5. (canceled)

7. The method of claim 1, wherein the nutritional supplement is administered from one to three times daily.

8. The method of claim 1, wherein the nutritional supplement is administered once a week.

9. The method of claim 1, wherein the nutritional supplement is administered on an empty stomach.

10. A method of increase linear growth in borderline GH deficient and non-GH deficient children, comprising:
providing an oral nutritional supplement, comprising:
L-arginine hydrochloride;
oxo-proline;
L-lysine hydrochloride; and
N-acetyl-L-cysteine, L-glutamine, or both;
wherein the L-arginine hydrochloride and the oxo-proline are present in a 1:1 weight ratio; and
orally administering the nutritional supplement to a borderline GH deficient and non-GH deficient child.

11. The method of claim 10, wherein the nutritional supplement is present in an amount of 2.9 grams.

12. The method of claim 10, wherein the nutritional supplement is in powder, tablet, capsule, liquid, or wafer form.

13. The method of claim 10, wherein the nutritional supplement is administered from one to three times daily.

14. The method of claim 10, wherein the nutritional supplement is administered once a week.

15. The method of claim 10, wherein the nutritional supplement is administered on an empty stomach.

16. The method of claim 10, further comprising L-glutamine.

17. The method of claim 10, further comprising schizonepeta powder.

18. A method of increase linear growth in borderline GH deficient and non-GH deficient children, comprising:
providing an oral nutritional supplement comprising an amino acid mixture, wherein the amino acid mixture consists essentially of:
L-arginine hydrochloride,
oxo-proline,
L-lysine hydrochloride, and
N-acetyl-L-cysteine, L-glutamine, or both; and
orally administering the nutritional supplement to a borderline GH deficient and non-GH deficient child.

19. The method of claim 1, wherein the L-lysine is present in an amount from 7 to 9 moles.

20. The method of claim 1, wherein the nutritional supplement is in powder, tablet, capsule, liquid, or wafer form.
